(19) United States
(12) Patent Application Publication
(10) Pub. No.: US 2023/0190885 A1
(43) Pub. Date: Jun. 22, 2023

(54) PLACENTAL ALKALINE PHOSPHATASE TO PROMOTE SCAR-FREE HEALING OF ACNE VULGARIS AND OTHER INFLAMMATORY SKIN LESIONS CAUSED BY MICROBIAL INFECTIONS

(71) Applicant: Zoltan Laboratories, LLC, Albert Lea, MN (US)

(72) Inventor: Zoltan Kiss, Austin, MN (US)

(21) Appl. No.: 17/556,675

(22) Filed: Dec. 20, 2021

(51) Int. Cl. A61K 38/46; A61K 45/06; A61K 9/00; A61P 31/22; C12N 9/16

(52) U.S. Cl. CPC A61K 38/465; A61K 9/0014; A61K 45/06; A61P 31/22; C12N 9/16; C12Y 301/03001

(57) ABSTRACT

This invention demonstrates that topically applied placental alkaline phosphatase dispersed in Vaselinum cholesterinatum promotes scar-free healing of acne vulgaris of bacterial origin. Other inflammatory skin conditions caused by bacterial infection and improved by placental alkaline phosphatase include nail infections as well as infections of non-healing diabetic wounds, puncture wounds, surgical wounds and incisions, burns, abrasions, lacerations, ulcers, gum disease, skin tears, or wounds caused by bites or stings. Placental alkaline phosphatase was also found to promote healing of skin lesions, with or without co-administering an antiviral drug, caused by reactivated Varicella zoster (Shingles), Herpes Simplex Virus-1 (oral herpes), or Herpes Simplex Virus 2 (vaginal/genital herpes). In all of these cases, placental alkaline phosphatase was found to significantly reduce irritation or pain associated with bacterial or viral infection and resulting skin damage.

### FIELD OF THE INVENTION

This invention demonstrates the promoting effects of topically applied placental alkaline phosphatase containing cream on scar-free healing of acne vulgaris and other inflammatory skin lesions primarily caused by microbial infection.

### BACKGROUND

### Acne Vulgaris

For practical reasons, many dermatologists still divide acne vulgaris into two major types: 1. Noninflammatory: Noninflammatory lesions are the open (blackheads) or closed (whiteheads) comedones. 2. Inflammatory: Inflammatory lesions include papules, pustules, and nodules. Most people have the so called non-inflammatory type. It should be noted that inflammation is also present in the “noninflammatory” acne, so the distinction between the two forms is often difficult.

The pathogenesis of acne vulgaris involves four major factors such as androgen-dependent sebogenesis, hyperkeratinization of the infundibulum, colonization by Propionibacterium acnes, or P. acnes (also called Cutibacterium acnes, or C. acnes), and inflammation. Acne vulgaris can arise when the follicles are blocked due to hyperkeratinization and formation of a plug of keratin (a protein that binds keratinocytes together), sebum (a waxy, fatty substance that is composed of triglycerides, wax esters, squalene, and free fatty acids produced by sebocytes), and dead epidermal cells. The plug may increase in size to form an open or closed comedo that then colonized by P. acnes and few other bacteria that may initiate inflammation that, with other factors involved, may lead to the formation of inflammatory lesions in the dermis around the comedo resulting in redness and often scarring. The lesions result in secretion of ATP from damaged cells resulting in worsening of inflammation.

Although there is no visible redness and irritation associated with early comedone formation, research has shown that inflammation at the microscopic level is present during all stages of acne formation. In fact, most recently acne is viewed as a chronic inflammatory disease that is triggered by inflammation often lasting for several years with patterns of relapse and remission, All clogged pores are triggered by inflammatory molecules, especially by a specific inflammatory molecule called interleukin-1. Interleukin-1 increases the body’s production of epidermal cells that in turn contributes to clogging the pores. Interestingly, in acne-prone people, even pores that were not actively developing into acne showed inflammation around the pore.

In addition to triggering clogged pores, inflammation can also make existing “non-inflammatory” acne worse. Once a comedone bursts, the sebum, skin cells, and bacteria that were in the comedone come into direct contact with the surrounding skin and trigger the body’s immune system. Then, the skin views the contents of the comedone as harmful invaders and fights the invasion by recruiting immune cells. These cells drive a wave of inflammation that causes the visible redness and pain associated with a papule, pustule, nodule, and/or cyst. This new wave of inflammation contributes to scars or hyperpigmentation forming dark/red spots [Williams, H. C., Dellavalle, R. P. and Garner, S. Acne vulgaris. Lancet 379, 361 - 372, 2012; Tanghetti, E. A. The role of inflammation in the pathology of acne. J. Clin. Aesthet. Dermatol. 6, 27-35, 2013; Saurat, J. Strategic targets in acne: the comedone switch in question. Dermatology 231, 105 - 111, 2015; Dreno, B., Gollnick, H.P.M., Kang, S. et al. Understanding innate immunity and inflammation in acne: implications for management. J. Eur. Acad. Dermatol. Venereol. 29, 3 - 11. 2015].

Treatments for acne include topical retinoids that with time normalize abnormal hyperkeratinization in the infundibulum and novel topical retinoids with anti-inflammatory properties. Topical and oral antimicrobials inhibit bacterial proliferation and reduce inflammation. Topical benzoyl peroxide (BPO) is effective in reducing the growth of P. acnes with some impact on hyperkeratinization in the infundibulum. Anti-androgens can regulate androgen metabolism, resulting in suppression of sebum excretion. Orally administered isotretinoin is currently the only agent that can affect all four main factors implicated in acne.

### Acne and Scar Tissue Formation

Acne primarily affects the face with many subjects experiencing some degree of scarring, the severity of which correlates to acne grade. Acne scars result from an altered wound healing response to cutaneous inflammation, with inflammatory cell infiltrates found in most atrophic scars. Aberrant production and degradation of collagen during the healing process leads to various types of acne scars. In 80 to 90 percent of cases, there is a net destruction of collagen in the dermis that results in atrophic scars. Less commonly, there is a net gain of collagen that results in hypertrophic or keloid scars [Georgios Kravvas and Firas Al-Niaimi. A systematic review of treatments for acne scarring. Part 1: Non-energy-based techniques. Scars Burn Heal. 3: 2059513117695312, 2017].

### Lipopolysaccharide (LPS) and Acne

Keratinocytes, melanocytes, and Langerhans cells in the epidermis can recruit dendritic cells, macrophages, natural killer cells and polymorphonuclear leukocytes. For quick recognition of a large variety of pathogenic organisms, these cells rely on well conserved pattern recognition receptors which are created by germline DNA and respond to pathogen-associated molecular patterns (PAMPs). PAMPs are shared by many pathogens, allowing the innate immune system to mount a fast response that is nonspecifically active against multiple challenges. An important family of pattern recognition receptors are known as toll-like receptors (TLRs); these receptors can initiate innate immune responses and later influence adaptive immune responses. TLRs are transmembrane proteins that respond to PAMPs such as bacterial cell wall components and genetic material. For example, TLR2 reacts to peptidoglycan from gram-positive bacteria while TLR4 is triggered by lipopolysaccharide from gram-negative bacteria. Activation of TLRs stimulates a variety of intracellular pathways that ultimately cause release of proinflammatory and immunomodulatory cytokines, including interleukins (ILs) and tumor necrosis factor alpha (TNFα) each playing important roles in the trigger and maintenance of inflammation in acne lesions [Dreno, B.,, Gollnick, H.P.M.,, Kang, S., Thiboutot, D., Bettoli,V., Torres, V. and Leyden, J. Understanding innate immunity and inflammation in acne: implications for management. European Academy of Dermatology and Venerology. 29 (Suppl. 4), 3-11, 2015].

There is accumulating evidence in support of a relationship between increased intestinal permeability and acne. One mechanism appears to be involved is the loss of intestinal bifidobacteria. Such loss is critical, because bifidobacteria plays a role in maintaining integrity of the intestine and thus prevent the efflux of lipopolysaccharide (LPS) endotoxins into the systemic circulation. The loss of bifidobacteria is mainly due to poor dietary choices such as high fat and sugar-loaded foods that leads to increased intestinal permeability, encroachment of LPS endotoxins through the intestinal barrier, which in turn leads to low-grade inflammation, oxidative stress, insulin resistance, and feeling of sickness. In one study, most adolescents with acne showed positive reaction to LPS in the blood implying that LPS has a role in maintaining inflammation in acne [Bowel, P.W. and Logan., C.A. Acne vulgaris, probiotics and the gut-brain-skin axis - back to the future? Gut Pathogens 3:1, 1-12, 2011, and references therein].

It may be relevant to the present disclosure that human alkaline phosphatases dephosphorylate LPS resulting in the formation of a nontoxic lipid A group with only one phosphate group left within the LPS molecule [Poelstra, K,, Bakker, W.W., Klok, A.P., Hardonk, J.M. and Meijer, K.D. A physiologic function for alkaline phosphatase: endotoxin detoxification. Lab. Invest. 76, 319-27, 1997; PoelstraK., Bakker, W.W., Klok, A.P., Kamps, A.J., M J Hardonk, J.M. and D K Meijer, K.D.. Dephosphorylation of endotoxin by alkaline phosphatase in vivo. Am J Pathol. 151, 1163-1169, 1997]. However, because of the physiological complexity of acne, it would be difficult to predict beneficial effects of alkaline phosphatase solely based on its ability to dephosphorylate LPS.

### The Role of Bacteria in Painful Inflammatory Skin Conditions Other Than Acne

A good example for the role of bacteria in an inflammatory skin condition is nail infection (paronychia). Most often paronychia is caused by Staphylococcus aureus or E. coli invading damaged skin There are two types of nail infection:

A: Acute paronychia. Symptoms appear within few hours or days and even with antibiotic treatment can last up to 6 weeks.

B: Chronic paronychia. Symptoms develop slowly and it usually lasts six weeks or longer. Effective and early treatment is very important to avoid deeper penetration of bacteria because if that happens sometimes it may result in the removal of the affected finger or toe to prevent systemic infection.

Both types of nail infections are usually painful. It is relevant to this disclosure that bacteria release ATP [Mempin, R.,, Tran, H., Chen, C., Gong, H., Kim Ho, K, and Sangwei Lu, S. Release of extracellular ATP by bacteria during growth. BMC Microbiology, 13, 301-314, 2013, and references therein] which cither starts or contributes to the inflammatory condition and pain in the infected lesion as reviewed later under the “Role of Extracellular ATP in Inflammation”. It should be noted that damaged cells in the inflamed lesion also release ATP. Next, ectonucleotidases will produce adenosine which is incorporated into bacteria to make ATP essential to their growth. This circle of producing more inflammation and pain may be broken by inactivating extracellular ATP without forming less adenosine as described in this disclosure.

Bacteria can infect any other damaged areas of skin (wounds) if not treated in time with antibiotics. Wounds may be puncture wounds, surgical wounds and incisions, burns, abrasions, lacerations, ulcers, gum disease, skin tears, or wounds caused by bites or stings. Nonhealing wounds, occurring frequently in diabetic patients, are also often become infected. In all these cases, damaged cells release ATP to the extracellular space contributing to inflammation and pain as described for nail infection. Should a drug be available to immobilize extracellular ATP and prevent or significantly reduce the formation of adenosine, it could be used proactively on wounds to prevent colonization by drug resistant bacteria that also need adenosine in the extracellular space for their growth. In fact, scientists continue to ring alarm bells about the risks associated with the misuse of antimicrobials and advocate for developing innovative treatments that can replace antimicrobial drugs. A drug that can significantly reduce adenosine formation from extracellular ATP may offer such innovative antimicrobial treatment.

### Adenosine as a Promoter of Epidermal Cell Proliferation in Vivo in Inflamed Tissues

The effects of adenosine on the proliferation of keratinocytes depends on whether the studies are performed in vitro or in vivo. In cell cultures, adenosine has been shown to exert inhibitory effects on the proliferation of normal keratinocytes [Cook, P.W., Ashton, N.M. and Pittelkow, M.R. Adenosine and adenine nucleotides inhibit the autonomous and epidermal growth factor-mediated proliferation of cultured keratinocytes. J. Invest. Dermatology. 104, 976-981, 1995; Brown, J., Cornell, K. and Cook, P.W. Adenosine- and adenine-nucleotide-mediated inhibition of normal and transformed keratinocyte proliferation is dependent upon dipyridamole-sensitive adenosine transport. J. Invest. Dermatol. 115, 849-859, 2000; Iizuka, H., Adachi, R., Koizumi, H., Aoyagi, T., Ohkawara, A. and Miura, Y. Effects of adenosine and 2′-deoxyadenosine on epidermal keratinocyte proliferation: Its relation to cyclic AMP formation. J. Biol. Chem. 82, 608-612, 1984].

However, in inflamed or wounded tissues adenosine has opposite effects. Thus, in such in vivo situations, adenosine was shown to enhance proliferation of endothelial as well as epidermal cells and thus promoting wound healing [Feoktistov, I., Biaggioni, I. and Cronstein, B.N. Adenosine receptors in wound healing, fibrosis, and angiogenesis. Handb. Exp. Pharmacol. 193, 383-397, 2009; Montesinos, M.C., Desai, A., Chen, J.F., Yee, H., Schwarzschild, M.A., Fink, J.S. and Cronstein, B.N. Adenosine promotes wound healing and mediates angiogenesis in response to tissue injury via occupancy of A_{2A} receptors. Am. J. Pathol. 160, 2009-2018, 2002; Braun, M., Lelieur, K. and Kietzmann, M. Purinergic substances promote murine keratinocyte proliferation and enhance impaired wound healing in mice. Wound Rep. Reg. 14, 152-16, 2006]. Thus, while promoting wound healing by adenosine is a positive physiological effect, in case of acne promotion of excess proliferation of epidermal cells contributes to hyperkeratinization. Since in inflammatory tissues the extracellular levels of both adenosine 5′ triphosphate (ATP) and its metabolic product adenosine are chronically high, in case of acne it is desirable to reduce the contents of both. This could be achieved by immobilizing ATP and thereby preventing or reducing its metabolism to adenosine. In this invention a method will be described to accomplish this important goal.

### Role of Extracellular ATP in Inflammation

Extracellular ATP has been recognized as a major driver of systemic inflammation with contributions to many unhealthy conditions ranging from increasing the intestinal permeability (resulting in the release of LPS among others) to diabetes and some cancers [Cauwels, A., Rogge, E., Vandendriessche, B., Shiva, B. and Brouckaert, P. Extracellular ATP drives systemic inflammation, tissue damage and mortality. Cell Death and Disease. 5, e1102; doi:10.1038/cddis.2014.70, 2014; Kurashima, Y., Kiyono, H. and Kunisawa, J. Pathophysiological Role of Extracellular Purinergic Mediators in the Control of Intestinal Inflammation. Mediators of Inflammation. Volume 2015, Article ID 427125, 8 pages http://dx.doi.org/10.1155/2015/42712]. ATP is released into the extracellular compartment from necrotic cells/tissues, bacteria, and activated immune cells via pannexin-1 and connexin hemichannels. They are then recognized by multiple purinergic P2X and P2Y receptors that then involved in the activation of the inflammasome NLRP3. While activation of NLRP3 is important for contributing to inflammation, there is evidence for the role of some other mechanism as well [Cauwels, A., Rogge, E., Vandendriessche, B., Shiva, B. and Brouckaert, P. Extracellular ATP drives systemic inflammation, tissue damage and mortality. Cell Death and Disease 5, e1102; doi:10.1038/cddis.2014.70, 2014].

Extracellular ATP is removed by conversion into adenosine in a two-step enzymatic process involving the ectonucleotidases CD39 (ecto-apyrase) and CD73. However, under pathological conditions, including inflammation and hypoxia, extracellular ATP levels may increase due to active release as well as passive leakage from damaged or dying cells, in combination with downregulation of ectonucleotidases. In case of acne, the rate of ATP hydrolysis to adenosine is not yet clear. What is clear, however, is that a successful anti-acne drug will have to offset the effect of extracellular ATP on inflammation and prevent significant formation of adenosine and thus reduce hyperkeratinization.

### Role of ATP in Pain

It has been known for quite a while that iontophoretic application of ATP elicits pain in the human skin [Hamilton, S.G., Warburton, J., Bhattacharjee, A., Ward, J. and McMahon, S.B. ATP in human skin elicits a dose-related pain response which is potentiated under conditions of hyperalgesia. Brain 123, 1238-1246, 2000]. Subsequent studies revealed that epidermal keratinocytes are important components of the sensory system responding to mechanical stress, changes in temperature, and chemical stimuli as well as osmotic pressure all resulting in pain sensation, and that purinergic receptors significantly contribute to such sensor functions of the human skin [Denda, M., Nakatani, M., Ikeyama, K., Tsutsumi, M and Denda, S. Epidermal keratinocytes as the forefront of the sensory system. Exp. Dermatol. 16, 157-161, 2007]. Recently, large number of other observations have provided evidence for the significant role of ATP in acute and chronic pain including neuropathic, inflammatory, and cancer pain as reviewed in [Inoue, K. P2 receptors and chronic pain. Purinergic Signalling 3, 135-144, 2007, and references therein; Burnstock, G. Purinergic P2 receptors as targets for novel analgesics. Pharmacol Therapeutics 110, 433-454, 2006, and references therein]. Several different purinergic receptors have been implicated in mediating pain, including the P2Y₁, P2Y₂, P2X₃, the heterodimer P2X₂/ P2X₃, and P2X₄ receptors [Huang, J., Zhang, X. and McNaughton, A. Inflammatory pain: The cellular basis of heat hyperalgesia. Current Neuropharmacology 4, 197-206, 2006; Tominaga, M., Wada, M. and Masu, M. Potentiation of capsaicin receptor activity by metabolotropic ATP receptors as a possible mechanism for ATP-evoked pain and hiperalgesia. Proc. Natl. Acad. Sci. USA 98, 6951-6956, 2001; North, R.A. P2X₃ receptors and peripheral pain mechanisms. J. Physiol. 554, 301-308, 2003; Inoue, K., Tsuda, M. and Koizumi, S. ATP receptors in pain sensation: Involvement of spinal microglia and P2X₄ receptors. Purinergic Signalling 1, 95-100, 2005].

### The Role of Herpes Viruses in Skin Diseases

The human alpha herpesviruses herpes simplex (HSV-1, HSV-2) and varicella zoster virus (VZV) establish persistent latent infection in sensory neurons for the life of the host. All three viruses have the potential to reactivate causing recurrent disease. Regardless of the homology between the different virus strains, the three viruses are characterized by varying pathologies. Thus, HSV typically infects the oral cavity (HSV-1) or vaginal/genital mucosa (HSV-2), but both viruses can infect at the skin, the eye, and other surfaces of the body. On the other hand, varicella zoster is responsible for Chickenpox.

Herpes zoster, or shingles, is an often-painful rash, caused by reactivation of latent varicella zoster virus from a previous infection. The rash consists of blisters that typically clears up within 3 to 5 weeks depending on the degree of severity. By one estimate, during their lifetime about 30% of Americans will develop herpes zoster, which translates into an estimated 1 million cases per year. The risk of shingles increases with increasing age; about half of all cases occur among people at age 60 or older. People who are immunosuppressed, including those with leukemia, lymphoma, and human immunodeficiency virus (HIV) infection, and people who receive immunosuppressive drugs, such as steroids and cancer chemotherapy, also are at greater risk of developing shingles.

From 2017, the best preventive treatment for shingles available is the Shingrix vaccine that can be given to 60 years old or older people. However, it should be noted that (a) Shingrix vaccine does not protect about 9% of people 70 years old or older, (b) about half of singles cases are in people younger than 60 years, (c) those who already have shingles cannot be treated with Shingrix, and (d) immunocompromised people cannot get the Shingrix vaccine. Considering all these exceptions for the use of Shingrix, it appears that many people with shingles need to receive a distinct drug when Shingrix cannot be used. In those cases, several antiviral medicines such as acyclovir, valacyclovir, and famciclovir are available to reduce the severity and length of the disease. These drugs cut the length of the disease approximately by half with no significant effect on pain. Considering the debilitating effect of shingles on the patient, it would be desirable to use a drug in combination with antiviral medicines that could further reduce the time needed for healing along with significant reduction of pain. Because of the similarities among the herpes viruses, it is reasonable to suggest that such drug would also be useful to treat the syndromes caused by HSV-1 and HSV-2.

For reducing moderate pain and irritation caused by singles, ibuprofen, antihistamines as well as numbing creams or patches containing lidocaine or capsaicin may be used. For more severe pain drugs like corticosteroids or local anesthetics among others are prescribed.

### SUMMARY OF THE INVENTION

In one embodiment, this invention demonstrates the utility of highly purified and recombinant forms of placental alkaline phosphatase (PLAP) dispersed in Vaselinum cholesterinatum or another suitable carrier for effective and essentially scar free healing of acne. Vaselinum cholesterinatum consists of about 1.5 wt.-% cholesterol, about 5.0 wt.-% cerae lanae, and about 93.5 wt.-% Vaselinum flavum. To simplify the text, in the following sections full length placental alkaline phosphatase is termed “PLAP”. Further, PLAP dispersed in Vaselinum cholesterinatum or any other carrier will be referred to as “PLAP cream”.

Newer assays had demonstrated that Vaseline, a petrolatum product, is not comedogenic [Kligman, A. M. Petrolatum is not comedogenic in rabbits or humans: A critical reappraisal of the rabbit ear assay and the concept of “acne cosmetic.” J Soc Cosmet Chem 47, 41 - 48, 1996]. It is also important that PLAP is spontaneously inserted into lipid membranes [Saslowsky, E.D., Lawrence, J., Ren, X., Brown, A.D., Henderson, M.R. and Edwardson, M.J. Placental alkaline phosphatase is efficiently targeted to rafts in supported lipid bilayers. J. Biol. Chem. 277, 26966-26970, 2002]. This can explain how it can cross inside the interior of the acne lesion.

Although acne cases are still in most cases classified by dermatologists as noninflammatory and inflammatory ones, in this invention no distinction between the two large groups is made based on relatively recent discoveries that inflammation is a significant contributor to all forms of acnes [Tanghetti, E. The Role of Inflammation in the Pathology of Acne. J. Clin. Aesthet. Dermatol. 6, 27-35, 2013, and references therein; Dreno, B., Gollnick, H.P.M., Kang, S. et al. Understanding innate immunity and inflammation in acne: implications for management. J. Eur. Acad. Dermatol. Venereol. 29, 3 - 11. 2015, and references therein]. It is relevant to this invention that PLAP contains, in addition to its catalytic site, a nucleotide-binding site that specifically binds ATP and other purine nucleotides while binding of ATP to the catalytic site is insignificant [Llinas, P., Stura, E.A., Menez, A., Kiss, Z., Stigbrand, T., Millan, J.L. and Le Du, M.H. Structural studies of human placental alkaline phosphatase in complex with functional ligands. J. Mol. Biol. 250, 441-451, 2005]. This suggests that some of the effects of PLAP in the acne lesion may be altered upon binding extracellular ATP. Conversely, the effects of extracellular free ATP may be altered in the presence of extracellular PLAP in the acne lesion. For example, PLAP would be expected to prevent binding of ATP to a purinergic receptor involved in maintaining inflammation and proliferation of keratinocytes in the acne lesion. ATP binding to PLAP would also prevent or reduce formation of adenosine resulting in reduced rate of proliferation of epidermal cells. However, it should be pointed out that prior to this invention no proof has been presented that there may be a connection between the ability of PLAP to bind ATP and reduced inflammation in PLAP treated tissues.

At first sight, using PLAP for treating acne may sound counterintuitive because a major goal in acne treatment is to initially reduce proliferation of keratinocytes, while PLAP was shown to enhance proliferation of keratinocytes [Kiss, Z. Use of placental alkaline phosphatase to promote skin cell proliferation. U.S. Pat. No. 7,964,188]. However, in this invention data will be presented to show that PLAP and ATP together inhibit the proliferation of epidermal cells and fibroblasts as well as PLAP greatly reduces formation of extracellular adenosine from extracellular ATP by these cells strongly suggesting that these mechanisms play significant roles in diminishing inflammation in acne. It is also reasonable to assume that at the skin reparation phase, when ATP and adenosine are present in the extracellular space at much lower concentrations, if any, PLAP helps regeneration of a healthy epidermal layer at the site of acne by stimulating proliferation of fibroblasts and epidermal cells.

In a second embodiment, the invention describes the positive effects of PLAP cream on nail infection and associated pain that imply a broader utility of PLAP in controlling the effects of bacteria on inflammation and pain in wounds (non-healing diabetic wounds, puncture wounds, surgical wounds and incisions, burns, abrasions, lacerations, ulcers, gum disease, skin tears, or wounds caused by bites or stings). In these cases, cutting off the resupply of adenosine for bacterial growth may contribute to the effects of PLAP.

In a third embodiment, the invention demonstrates the utility of PLAP in enhancing the healing effects of antiviral drugs on shingles including increased pain relief. While the exact mechanism remains to be proven, virus infected cells are known to release ATP that most likely will promote inflammation. PLAP then may act, at least in part, via preventing the actions of ATP on stimulating the formation if inflammatory mediators by fibroblasts and epidermal cells. Regardless of the exact mechanism of PLAP action on shingles, based on the similarities of varicella zoster, HSV-1 and HSV, it is likely that PLAP also will be effective in promoting healing of lesions caused by HSV-1 (primarily oral cavity) and HSV-2 (primarily vaginal mucosa) infections.

In all cases, data also will be presented to show that PLAP can improve lesions caused by these viruses even without using antiviral drugs.

### DETAILED DESCRIPTION OF THE DRAWINGS

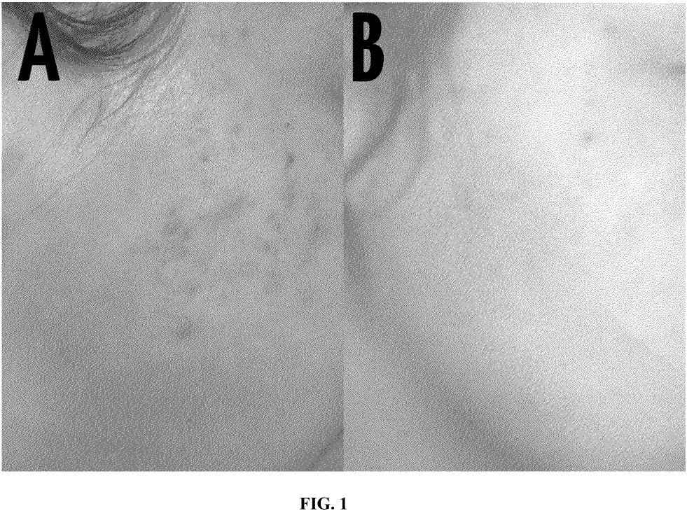

FIG. 1 shows a section of right side of face of a 16-year-old female subject before treatment (A) and after daily treatment with PLAP cream for 3 weeks (B). Before PLAP treatment, the acne covered area was treated with benzoyl peroxide as prescribed for 2 months with only small visible positive effects seen.

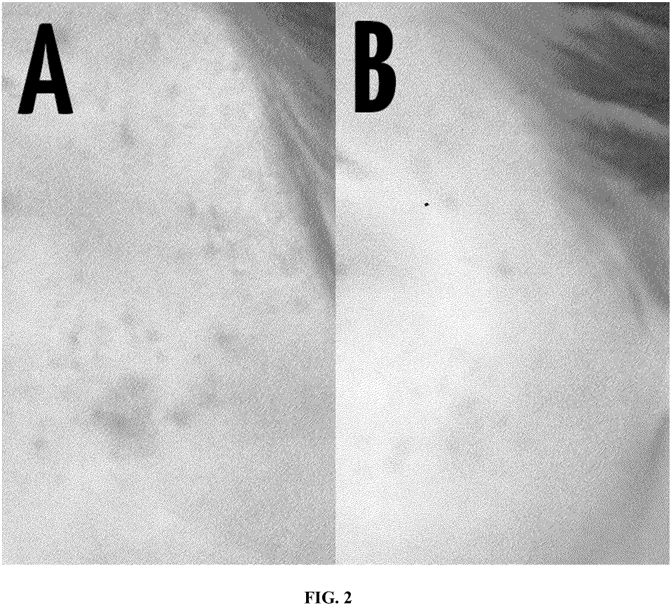

FIG. 2 shows a section of left side of face of the same female subject shown in FIG. 1 before treatment (A) and after daily treatment with PLAP cream for 3 weeks (B). Before PLAP treatment, the acne covered area was treated with benzoyl peroxide as prescribed for 2 months with only small visible positive effects seen.

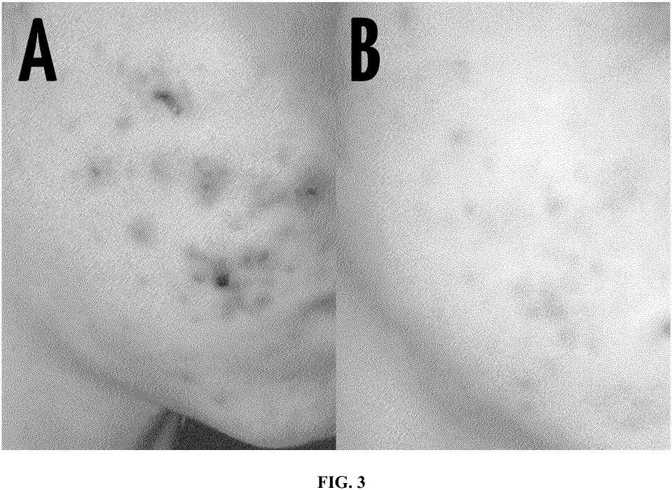

FIG. 3 shows a section of right side of face of a 17-year-old male subject before treatment (A) and after daily treatment with PLAP cream for 3 weeks (B). Before PLAP treatment, the acne covered area was treated with benzoyl peroxide as prescribed for 3 months with only small visible positive effects seen.

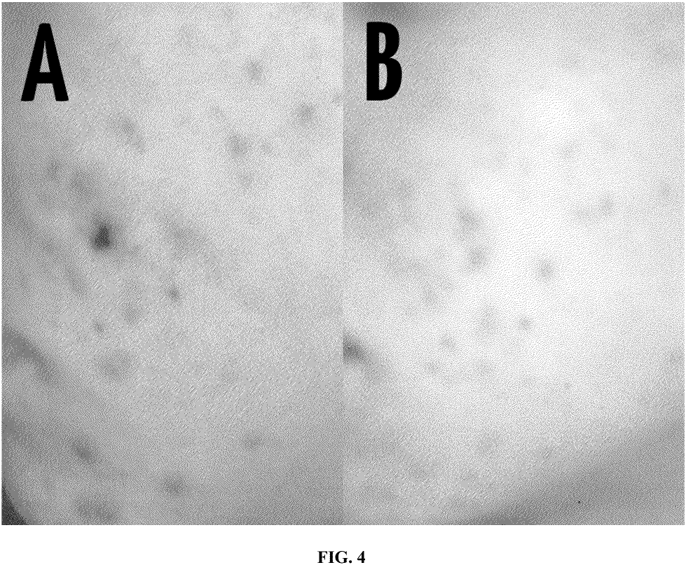

FIG. 4 shows a section of left side of face of the same male seen shown in FIG. 3 before treatment (A) and after daily treatment with PLAP cream for 3 weeks (B). Before PLAP treatment, the acne covered area was treated with benzoyl peroxide as prescribed for 3 months with only small visible positive effects seen.

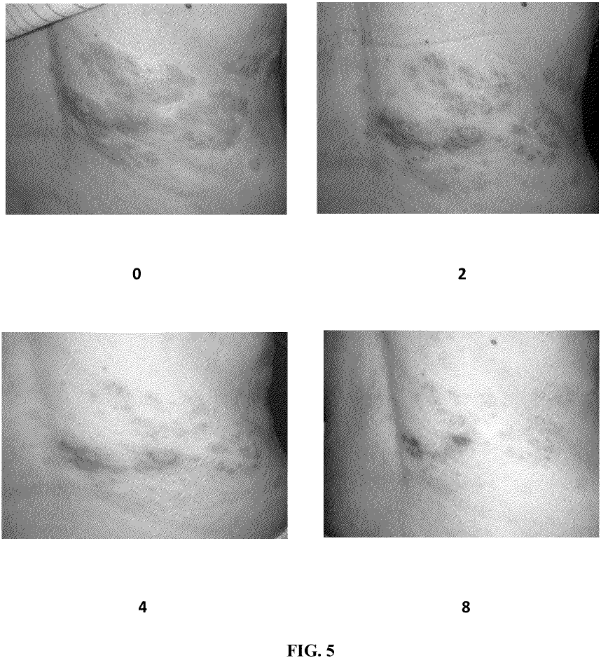

FIG. 5 demonstrates that combined daily treatment of a 72-year-old female subject with PLAP containing cream and acyclovir (four times daily as prescribed) for eight days helped to greatly reduce her frequently recurring shingles on her back.

### DETAILED DESCRIPTION

### A. The Active Agent

The active agent is full length placental alkaline phosphatase (PLAP) or an active derivative of PLAP. The alkaline phosphatase group of enzymes hydrolyze phosphate-containing compounds at alkaline pH. Mature PLAP is a dimer of two identical glycosylated subunits. Each subunit has an approximate molecular weight of 66 kDa, as determined by gel electrophoresis. As used herein, the phrase “active derivative of PLAP” means a sequence specifically derived from PLAP produced by a recombinant, enzymatic, or chemical method that can promote healing of acne. The recombinant active derivative (rPLAP) has catalytic alkaline phosphatase activity and contains at least 80% of the sequence of the full length PLAP.

Both PLAP and rPLAP; stimulate proliferation of skin fibroblasts and epidermal cells [Kiss, Z. U.S. Pat. 7,374,754, titled: Use of placental alkaline phosphatase to promote skin cell proliferation] which is important with respect to the healing phase of acne.

In the present application, for human use PLAP was highly purified from commercial PLAP (Sigma-Aldrich) as described in detail earlier [Kiss, Z. U.S. Pat. 7,374,754, titled: Use of placental alkaline phosphatase to promote skin cell proliferation]. The rPLAP used for in vitro studies with skin cells were prepared using a previously described method [Kozlenkow, A., Manes, T., Hoylaerts, M.F. and Millan, J.L. Function assignment to conserved residues in mammalian alkaline phosphatase. J. Biol. Chem. 277, 22992-22999, 2002].

Other recombinant methods to obtain quantities of PLAP (and active derivative) are also suitable. Since cDNA of PLAP is available, recombinant protein can be produced by one of the existing conventional methods for recombinant protein expression as reported by others [Beck, R. and Burtscher, H. Expression of human placental alkaline phosphatase in Escherichia coli. Protein Expression and Purification 5, 192-197, 1994; Heimo, H., Palmu, K. and Suominen, I. Human placenta alkaline phosphatase: Expression in Pichia pastoris, purification and characterization of the enzyme. Protein Expression and Purification 12, 85-92, 1998].

Bacterial expression yields non-glycosylated PLAP. So far there is no reported evidence that the biological effects of native glycosylated PLAP and bacteria produced PLAP would be significantly different. Thus, in the methods of the present invention native glycosylated PLAP and its active derivatives as well as non-glycosylated PLAP and its active derivatives may be used interchangeably.

If placenta derived PLAP preparation is to be used in the practice of the present invention, a raw extract should be treated to enrich the concentration of PLAP and obtain a highly purified preparation. A highly purified preparation will have a much higher concentration of the active component than found in a raw tissue extract. A highly purified PLAP preparation does not contain detectable amounts of other proteins or other impurities, or it contains such minimum amounts of reliably identified contaminants that the benefits of using such preparation far out-weight the accompanying potential risks. A highly purified PLAP preparation obtained from a starting placenta derived material by several purification steps (such as solvent extraction, column separation, chromatographic separation, etc.) that enrich the concentration of PLAP, relative to the starting material, to an extent that PLAP is the dominating component, and the remaining components do not pose any significant health risk and do not reduce the beneficial effects of PLAP. The term “highly purified” should not be construed to connote absolute (100%) purity.

### B Methods of Use

### Local Treatment of Acne With PLAP Cream

Highly purified placenta derived PLAP, or essentially pure recombinant PLAP, or an essentially pure active derivative thereof, is dispersed in a suitable carrier and gently massaged onto the acne lesion. The carrier used in the invention is Vaselinum cholesterinatum, but it may be replaced by Vaselinum flavum or Vaselinum album. Vaselinum cholesterinatum consists of about 1.5 wt.-% cholesterol, about 5.0 wt.-% cerae lanae, and about 93.5 wt.-% Vaselinum flavum. In this invention, for a preparation containing PLAP or rPLAP in a carrier the term “PLAP cream” is used.

Other appropriate forms of PLAP containing compositions may contain other suitable carriers in the forms of gels, lotions, unguents, emollients, colloidal dispersions, suspensions, emulsions, oils, sprays, liposomes, non-ionic detergents, foams, mousses, and the like as used in the skin care industry with no damaging effects on the skin. Preferably, the chosen carrier would enable PLAP to exert its antiacne effect or at least would not reduce it. In addition, the carrier should not be comedogenic and should not inhibit interaction of PLAP with the acne lesion. In the preparation of the carrier-PLAP mixture, PLAP is first dissolved in distilled water or a suitable buffer and then mixed with the carrier. The PLAP cream contains highly purified PLAP or rPLAP, in the amount of 0.1 to 10 mg per one gram carrier gently massaged onto the area of acne lesion. The amount of PLAP depends on the severity of acne. In cases of light, moderate and severe acnes the recommended amount of PLAP per 1 gram carrier is 0.1 to 0.5 mg, 0.6 to 2 mg, and 2.1 to 10 mg PLAP, respectively. Before each daily treatment the acne lesion needs to be cleaned with a facial cleanser as recommended by the manufacturer. The length of the daily treatments is typically for about 3 weeks, but it may be extended until the lesion is healed.

Therapeutically effective amounts of recombinant PLAP or other active derivatives of PLAP, may also be employed as the active components for the treatment of acne with the same recommended amounts as indicated for PLAP. The term “active” means that the given PLAP derivative exerts antiacne effects comparable to that of the highly purified PLAP. The term “therapeutically effective amount” in this specification indicates a dosage of PLAP or an active derivative that is effective in healing the acne lesion.

In some embodiments, the PLAP cream may include one or more well tested additives or enhancers from a list offered by pharmaceutical companies including preservatives, biologically active compounds with positive effects on the recovery of normal skin texture, buffers, moisture-control compounds, or antibiotics, for example. In other embodiments, the composition essentially contains the carrier and PLAP. As used here, the phrase “essentially contains” means that the given composition has no other ingredient in it in addition to PLAP or an active derivative and a carrier.

The PLAP cream can be made using several suitable techniques. In some embodiments, PLAP, optional additives, preservatives, antibiotics, and enhancers as well as a carrier are mixed within a commercial mixer to form a gel or the like. All conventional methods known in the art for mixing may be suitable. Various equipment is also available to manufacture liposomal preparations (which provides for controlled, sustained release of the components). In pharmaceutical composition embodiments, methodologies for the formulation are well known, and can be found, for example, in Remington’s Pharmaceutical Sciences, Eighteenth Edition, A.R. Gennaro, Ed., Mack Publishing Co. Easton, PA 1990, incorporated hereby by reference. Since PLAP activity remains stable by heating the PLAP cream for 30 min at 65 Celsius, such heating step may be included in the preparation of the final formulation for sterilization purpose.

### Local Treatment of Bacterial Skin Infections Other Than Acne With PLAP Cream

PLAP cream can also be used for the local treatment of (a) nail infections, (b) already infected wounds along with antibiotics, and (c) wounds infected with drug resistant bacteria. The same carriers and additives may be used as described above for the acne treatment, The recommended amount of PLAP per 1 gram carrier is 1.0 to 10 mg. Generally, the larger the lesion or wound the more PLAP is needed. As examples, if the area of the lesion is 1 cm² and the wound area is 10 cm², then the recommended concentration of PLAP is 1 mg and 5 mg per 1 gram carrier, respectively. In case of nail infection, on the first day the treatment is repeated hourly for 3 hours followed covering the lesion. The treatment is repeated on the second day and if still needed on the third day. Treatment of nail infections with PLAP cream does not require co-treatment with antibiotics. The infected wounds are treated with PLAP cream as nail infections except that it is combined with antibiotics as prescribed. In cases when infected wounds do not respond to antibiotics, meaning they were infected by drug resistant bacteria, they are treated with PLAP cream daily without treatment with antibiotics.

### PLAP Cream for Local Treatment of Skin Infected With Reactivated Varicella Zoster, Herpes Simplex Virus-1 (HSV-1) or Herpes Simplex Virus 2 (HSV-2)

The PLAP cream can be used for the local treatment of Shingles or other skin lesions caused by reactivated HSV-1 or HSV-2 generally, but not always, along with treatment with a prescribed antiviral drug. The same carriers and additives may be used as described for the acne treatment, The recommended amount of PLAP per 1 gram carrier is 2.0 to 10.0 mg. Generally, the larger the lesion the more PLAP per 1 gram carrier is needed. As examples, if the area of the lesion is 10 cm² or smaller, 2 mg per 1 gram carrier is suitable. If the area of the lesion is larger than 10 cm² then, depending on the lesion’s size, the recommended concentration of PLAP is between 2.1 to 10.0 mg per 1 gram carrier. Local treatment of the lesion with PLAP cream is performed once daily along with oral administration of the prescribed antiviral drug (usually four-times a day). Local administration of the antiviral drug is not recommended. In case of shingles, when the lesion treated with placental alkaline phosphatase or an active derivative is smaller than 2.0 cm², the use of an antiviral drug is optional. In cases of skin lesions caused by HSV-1 or HSV-2, the use of an oral antiviral drug is optional regardless of the lesion’s size.

### EXAMPLES

### Example 1 Production, Purification, and Spectrophotometric Assay of PLAP and rPLAP

A purification procedure consisting of several steps was performed to further purify the commercially obtained PLAP and to yield a homogeneous band in electrophoretic separation. The same purification procedure and assay was followed that had been described in detail elsewhere [She, Q.-B., Mukherjee, J.J., Huang, J.-S., Crilly, K.S. and Kiss, Z. Growth factor-like effects of placental alkaline phosphatase in human and mouse embryo fibroblasts. FEBS Lett., 469, 163-167, 2000].

Recombinant wild-type PLAP (rPLAP) was produced exactly as described by Kozlenkov et al. [Kozlenkov, A., Manes, T., Hoylaerts, M.F. and Millan, J.L. Function assignment to conserved residues in mammalian alkaline phosphatases. J. Biol. Chem. 277, 22992-22999, 2002].To simplify the recovery and purification of rPLAP, the glycosylphosphatidylinositol anchoring sequence of PLAP was replaced by the FLAG octapeptide, and rPLAP was expressed as secreted, epitope-tagged, enzyme. rPLAP is an example of an active derivative of full length PLAP. It demonstrates that active derivatives of full length PLAP with altered and shorter sequences can be made using recombinant methods.

### Example 2 Treatment of Acne Lesions on the Right Side of Face of a 16-Year-Old Female With PLAP Cream

FIG. 1 shows a section of right side of the face of a 16-year-old female subject with moderate acnes at multiple spots before treatment (A) and after daily treatment with PLAP cream (3-mg PLAP in 1-gram Vaselinum cholesterinatum) for 3 weeks (B). Before PLAP treatment, the acne covered area was treated with benzoyl peroxide as prescribed for 2 months that showed only small visible positive effects. In contrast, treatment with PLAP for 3 weeks was sufficient to heal the acne lesion by about 90% without visible scar tissue. Complete recovery of the original skin texture required about 5 weeks with no additional treatments needed between 3 to 5 weeks. The sites of the healed acne lesions have remained free of visible scar tissue since then (15 years).

### Example 3 Treatment of Acne Lesions on the Left Side of Face of a 16-Year-Old Female With PLAP Cream

FIG. 2 shows a section of left side of the face of the same female subject shown in FIG. 1 before treatment of moderate acnes at multiple spots (A) and after daily treatment with PLAP cream (3-mg PLAP in 1-gram Vaselinum cholesterinatum) for 3 weeks (B). Before PLAP treatment, the acne covered area was treated with benzoyl peroxide as prescribed for 2 months that showed only small visible positive effects. In contrast, treatment with PLAP for 3 weeks was sufficient to heal the acne lesion by about 80-85% without visible scar tissue. Complete recovery of the original skin texture required about 6 weeks with no additional treatments needed between 3 to 6 weeks. The sites of the healed acne lesions have remained free of visible scar tissue since then (15 years).

### Example 4 Treatment of Acne Lesions on the Right Side of Face of a 17-Year-Old Male With PLAP Cream

FIG. 3 shows a section of right side of face of a 17-year-old male with multiple severe acnes before treatment (A) and after daily treatment with PLAP cream (5-mg PLAP in 1-gram Vaselinum cholesterinatum) for 4 weeks (B). Before PLAP treatment, the acne covered area was treated with benzoyl peroxide as prescribed for 3 months with only small visible positive effects. In contrast, treatment with PLAP for 4 weeks was sufficient to heal the acne lesion by about 75-80% without visible scar tissue. Complete recovery of the original skin texture required about 8 weeks with no additional treatments needed between 4 to 8 weeks. The sites of the healed acne lesions have remained free of visible scar tissue since then (16 years).

### Example 5 Treatment of Acne Lesions on the Left Side of Face of a 17-Year-Old Male With PLAP Cream

FIG. 4 shows a section of left side of face of the same male shown in FIG. 3 before treating his multiple severe acnes (A) and after daily treatment with PLAP cream (5-mg PLAP in 1-gram Vaselinum cholesterinatum) for 3 weeks (B). Before PLAP treatment, the acne covered area was treated with benzoyl peroxide as prescribed for 3 months with only small visible positive effects. In contrast, treatment with PLAP for 3 weeks was sufficient to heal the acne lesion by about 75-85% without visible scar tissue. Complete recovery of the original skin texture required about 6 weeks with no additional treatments needed between 3 to 6 weeks. The sites of the healed acne lesions have remained free of visible scar tissue since then (16 years).

### Example 6 Additional Observations on Acne Treatment

The examples shown in FIGS. 1-4 and additional similar observations with one female and two male adolescents, each with severe form of acne, strongly suggested that early treatment of acnes with PLAP would result in faster healing. This idea was tested with 16-17-year-old males who started to develop acne during a 2 to 4-day period. Treatments (2-mg PLAP in 1-gram Vaselinum cholesterinatum) were applied at bedtime only once. In each case the acne was completely healed within 2-5 days without any scar tissue formation.

### Example 7 Nail and Wound Infections

Six subjects with painful fingernail infection, each rapidly developed within 24 hours of the initial skin damage, were treated with PLAP cream once within hours of detecting the signs of infection. In each case, the pain subsided within about 30 minutes but at a lower level it soon returned. Then, an additional treatment was applied after about one hour of the first treatment which completely abolished the pain. The infection was resolved within 24-48 hours without further treatment. Although no controls could be included, according to medical records nail infections rarely, if ever, resolve within such a short time.

On the basis of PLAP’s effects on nail infection, PLAP is fully expected to exert beneficial anti-inflammatory effects on any kind of other wounds infected with bacteria. Such use of PLAP would be especially beneficial if the wound is infected with drug resistant bacteria. Since PLAP strongly reduces formation of adenosine from extracellular ATP that would otherwise support bacterial growth, PLAP will likely curb the growth of drug resistant bacteria. This is an extremely important aspect of PLAP effect considering that in the U.S. every year nearly 100,000 people die because of wound infection including deaths due to diabetic non-healing infected wounds.

### Example 8 PLAP Promotes Healing of Shingles

### Shingles With Serious Symptoms

Two individuals (one female and one male) had recurrent painful shingles at 10-16 months intervals, usually lasting for 4-6 weeks without treatment, associated with pain lasting beyond 6 weeks that prevented them to get a good night sleep. When acyclovir became available, both subjects received daily (4-times a day) oral acyclovir treatment during the last 3 cycles of recurrence which resulted in about 40-50% remission in 10-12 days and full healing by the end of third week. Acyclovir had no significant effect on pain.

When a new recurrence of shingles occurred again, the same two subjects were co-treated with local PLAP cream (5 mg PLAP in 1-gram Vaselinum cholesterinatum) and oral acyclovir. The two subjects responded to this combination treatment similarly.

FIG. 5 demonstrates the combined effects of PLAP cream and acyclovir on the female subject (72 years old). She started the treatment with acyclovir and PLAP cream on day 0. As shown, the combined treatment reduced the size of the lesion below 50% after 4 days of treatments. By day 8, the size of the lesion was only about 10-15% of the original size, and this was accompanied by significant reduction in pain which allowed her to sleep, from day 1 during each consecutive night, at least 6 hours without waking up. Complete healing of both subjects occurred by day 12 (the female subject) and day 14 (the male subject). Thus, combination treatments with PLAP and acyclovir (and most probably with other antiviral agents as well) yield significantly better results on shingles than treatments with acyclovir alone.

### Shingles With Moderate Symptoms

Two female individuals, 32- and 61- year-old, respectively, had recurrent (practically yearly) moderate shingles (one had under the breast, the other had on the arm, both approximately the size of 1-2 cm²) previously lasting for about 2-3 weeks. In both cases, the shingles were associated with well noticeable irritation. After the first treatment with PLAP cream ((2.0 mg PLAP in 1-gram Vaselinum cholesterinatum) without acyclovir the irritation became unnoticeable within 1 hour and the shingles disappeared by around the 3rd and 4th day in the cases of the 32=year-old and 61-year-old woman, respectively. This indicates that shingles of moderate size can be effectively treated with PLAP without using an antiviral medication.

### Herpes (Oral)

Three female subjects having recurrent oral herpes with 6-12 months frequency during the last 3 years usually lasting for 4-6 days and accompanied by irritation or mild pain were treated once with PLAP cream (2.0 mg PLAP in 1-gram Vaselinum cholesterinatum) without an antiviral drug. In each case, the irritation was greatly reduced within one hour and the skin healed within 2 days.

### Herpes (Genital)

Two male subjects having recurrent relatively small size (0.4-0.6 cm²) but moderately painful genital herpes, lasting for 10-16 days, were treated with PLAP cream (2.0 mg PLAP in 1-gram Vaselinum cholesterinatum) without an antiviral drug on two consecutive days. Pain was significantly reduced in a lasting manner within one hour of the first treatment, and the lesions resolved/healed in 3 days (0.4 cm² lesion) and 5 days 0.6 cm² lesion.

### Example 9 Effects of PLAP, rPLAP, and ATP on the Proliferation of Human Epidermal HaCaT Cells and Human Skin Fibroblast Cell Line CCD 966 SK

The immortalized human HaCaT keratinocyte cell line, isolated in 1988 [Boukamp, P., Petrussevska, R.T., Breitkreutz, D., Hornung, J., and Markham, A., “Normal keratinization in a spontaneously immortalized aneuploid human keratinocyte cell line,” J. Cell. Biol., 106, 761-771, 1988], was provided for these studies by the Institute of Dermatology, Szeged University, , Szeged, Hungary). HaCaT cells were maintained in 10% fetal bovine serum (FBS) and 1% penicillin-streptomycin -containing Dulbecco’s modified Eagle’s medium (DMEM) up to 80% confluency in an incubator (5% CO₂, 37° C.). Cells were plated into 96 well plates at 20% confluency and incubated for 72 hours in an incubator (5% CO₂, 37° C.) with 200 nM purified PLAP or rPLAP in the absence or presence of 100 µM ATP.

CCD 966 SK fibroblasts, originally derived from a 52-year-old subject and purchased from American Type Culture Collection (Alexandria, Virginia), were at passage 5 when used. The fibroblasts were maintained and treated as described above for the HaCaT cells.

The relative changes in the number of viable cells were determined by the MTT assay. This colorimetric assay is based on the ability of living healthy cells (mostly the mitochondrial compartment), but not dead cells, to reduce 3-(4,5-dimethyl thizaol-2-yl)-2,5-tetrazolium bromide to a blue formazan product [Carmichael, J., De Graft, W.G., Gazdar, A.F., Minna, J.D. and Mitchell, J.B. Evaluation of tetrazolium-based semi-automated colorimetric assay: Assessment of chemosensitivity resting. Cancer Res. 47, 936-942, 1987] This technique is a widely used and accepted method to accurately determine the relative numbers of viable cells. For example, this is the official method used by the National Cancer Institute to screen anticancer drugs. In most cases, when the test agent does not strongly influence the oxidation-reduction balance of cells, the MTT assay is essentially a proliferation assay.

A MULTISKAN MS microplate reader purchased from Labsystems (Franklin, Massachusetts) was used to measure the formation of formazan as an increase in absorbance at a test wavelength of 540 nm and a reference wavelength of 690 nm. In the data analysis, data were expressed as mean values ± standard deviation (S.D.) of eight incubations (from eight wells) in one experiment. In each case, incubations were performed for 72 hours.

As data in TABLE 1 shows, ATP alone had no effects on the proliferation of either cell line, while both PLAP and rPLAP clearly stimulated proliferation of both cell lines. It is noteworthy that in the absence of ATP the stimulatory effects of PLAP and rPLAP were of similar magnitude. In contrast, in the presence of ATP both PLAP nor rPLAP inhibited the proliferation of epidermal cells and fibroblasts.

These results strongly indicate that depending on the concentration of extracellular ATP, PLAP and rPLAP will exert two different effects on skin cell proliferation. In the presence of ATP in the extracellular space (like in inflamed tissues), PLAP and rPLAP will inhibit proliferation of skin cells, while in the absence of ATP in the extracellular space (in the regenerative phase) PLAP and rPLAP will stimulate proliferation of skin cells thereby helping to restore the damaged skin.

TABLE 1

Combined effects of PLAP and rPLAP on the Proliferation of HaCaT and 966 SK Fibroblasts

| Treatment | Viability of Cells (Absorption: A₅₄₀) | |
|---|---|---|
| | HaCaT cells | CCD 966 SK |
| None, 0 hour | 0.512±0.033 | 0.411±0.023 |
| None, 72 hours | 0.859±0.052 | 0.705±0.035 |
| ATP, 72 hours | 0.841±0.037 | 0.686±0.029 |
| PLAP, 72 hours | 1.086± 0.047 | 0.853±0.040 |
| PLAP + ATP, 72 hours | 0.736±0.038 | 0.590±0.017 |
| rPLAP, 72 hours | 1.133±0.049 | 0.818±0.032 |
| rPLAP + ATP, 72 hours | 0.715±0.020 | 0.597±0.039 |

### Example 10. Determination of ATP Hydrolysis in Intact HaCaT Human Epidermal Cells and CCD 966 SK Human Fibroblasts

In these experiments, PLAP was incubated with human HaCaT cells and human 966 SK fibroblasts. The plasma membranes of both cell lines contain ectonucleotidases that hydrolyze ATP to result in the formation of adenosine. If PLAP binds ATP as reported earlier, it will compete with the ectonucleotidases for ATP. In this case, in the presence of PLAP the formation of adenosine, the final product of ATP hydrolysis, should be reduced. The experiment performed under this Example served to examine this possibility.

HaCaT and 966 SK cells were grown in 10% serum containing DMEM medium (0.1 ml) in 96-well plates to confluence. First, the medium was changed for serum-free medium. Then, selected wells received 200 nM purified PLAP 5 min prior to the addition of 2,000,000 c.p.m. (0.1 mM) of [U-¹⁴C] ATP (purchased from Amersham). Incubations in an incubator were performed for 2 hours. Fifty ml aliquots were withdrawn into small centrifuge tubes followed by the addition of 0.15 ml of chloroform methanol 2:1 mixture containing 1 mM adenosine (to help UV visualization and reduce cellular uptake of [U-¹⁴C] Adenosine) to precipitate the proteins and eliminate lipids. After centrifugation, 5 µl aliquots of the lower phase were transferred to fluorescent PEI-cellulose (polyethyleneimine-impregnated cellulose) plates, and after drying the plates they were developed with water in a flat bottom chamber. After drying and visualizing the adenosine and nucleotide spots under UV (all nucleotides remained in the application spots), the respective areas were cut out, placed in scintillation vials, and radioactivity was measured in a liquid scintillation spectrometer. Data is the mean of three independent determinations (from three separate wells) ± S.D. in the same experiment.

The data, shown in TABLE 2, demonstrates that in both cell lines PLAP greatly reduced the hydrolysis of ATP and related nucleotides (i.e., more radiolabeled ATP and related nucleotides remained in the medium) which coincided with dramatic reduction in the formation of radiolabeled adenosine. The difference between the ATP lost and adenosine present in the medium most probably reflects uptake of a portion of newly formed adenosine into cells which was not measured in this experiment.

The best interpretation of data is that PLAP, by binding ATP, competed with ectonucleotidases to gain access to this substrate. One major consequence of this event is that less adenosine is formed, as shown in TABLE 2.

TABLE 2

PLAP reduces ATP hydrolysis and adenosine formation in HaCaT and CCD 966 SK cell cultures

| Treatment | Total [U-¹⁴C] nucleotide | [U-¹⁴C] Adenosine |
|---|---|---|
| | HaCaT cells; | c.p.m./well |
| 0 minute | 1,711,445 ± 50,034 | 953 ± 151 |
| 2 hours; None | 1,009,901 ± 44,604 | 548,050 ± 23,477 |
| 2 hours; PLAP, 500 nM | 1,572,853 ± 33,737 | 71,048 ± 2,220 |
| | CCD 966 SK cells; | c.p.m./well |
| 0 minute | 1,771,762 ± 34,405 | 1,116-963-875 |
| 2 hours; None | 1,295,488 ± 48,371 | 372,989 ± 25,283 |
| 2 hours; PLAP, 500 nM | 1,653,949 ± 42,791 | 34,019 ± 4,892 |

1. A method for promoting healing of acne and other skin lesions caused by bacterial infection comprising the step of topically administering to an area of the skin lesion a composition comprising a therapeutically effective amount of human placental alkaline phosphatase, or an active derivative thereof, dispersed in a suitable carrier.

2. The method of claim 1, wherein the other skin lesions include nail infections and infected wounds including non-healing diabetic wounds, puncture wounds, surgical wounds and incisions, burns, abrasions, lacerations, ulcers, gum disease, skin tears, or wounds caused by bites or stings.

3. The method of claim 1, wherein the amount of placental alkaline phosphatase or an active derivative thereof in the topically administered composition is 0.1 to 0.5 mg, 0.51 to 2 mg, and 2.1 to 10 mg PLAP per one gram carrier applied to light, moderate, and severe acne lesions, respectively.

4. The method of claim 2, wherein the amount of placental alkaline phosphatase or an active derivative thereof in the topically administered composition is 1.0 to 10.0 mg per 1 gram carrier.

5. The method of claim 4, wherein the amount of placental alkaline phosphatase or an active derivative thereof in the topically administered composition is 1 mg and 5 mg per 1 gram carrier when the size of the infected lesion is 1 cm² and 10 cm², respectively with every two cm² increase in the infected area resulting in the addition of 1 mg placental alkaline phosphatase per 1 g carrier.

6. The method of claim 1 wherein topical treatment of acne is applied daily as long as needed.

7. The method of claim 2, wherein in case of nail infection, on the first day the treatment is repeated hourly for 3 hours followed covering the lesion. The treatment is repeated on the second day and if still needed on the third day.

8. The method of claim 2, wherein topical treatment of infected wound applied daily as long as needed.

9. A method for promoting healing of skin lesions caused by reactivated Varicella zoster (Shingles), Herpes Simplex Virus-1 (oral herpes), or Herpes Simplex Virus 2 (mostly vaginal/genital herpes) comprising the step of topically administering to an area of the skin lesion a composition comprising a therapeutically effective amount of human placental alkaline phosphatase, or an active derivative thereof, dispersed in a suitable carrier.

10. The method of claims 9, wherein. the amount of placental alkaline phosphatase or an active derivative thereof is 2.0 mg and between 2.1 to 10.0 mg per 1 gram carrier when the lesion is 10.0 cm² or smaller and when the lesion is larger than 10 cm², respectively.

11. The method of claim 9, wherein the skin lesion is Shingles, which is also treated with an orally administered antiviral drug, for example acyclovir, valacyclovir or famciclovir, as prescribed, if the lesion is larger than 2.0 cm²; if the lesion is smaller than 2.0 cm², treatment with an orally administered antiviral drug is optional.

12. The method of claim 9, wherein co-treatment of the skin lesions caused by reactivated Herpes Simplex Virus-1 or Herpes Simplex Virus 2 with an oral antiviral drug is optional regardless of the lesion’s size.

13. The method of claims 9 to 12, wherein the treatments with topical placental alkaline phosphatase or an active derivative are administered once daily until the lesion is healed.

14. The method of claim 1, wherein the suitable carrier is Vaselinum cholesterinatum.

15. The method of claims 1 and 9, wherein the suitable carrier is Vaselinum, flavum, Vaselinum albaum, a gel, a lotion, an unguent, an emollient, a colloidal dispersion, a suspension, an emulsion, an oil, a spray, a liposomal preparation, a non-ionic detergent, foams, mousses, and the like as used in the skin care industry that are safe to use on the skin.

16. The method of claims 1 and 9, wherein placental alkaline phosphatase or an active derivative thereof effectively reduces pain and irritation caused by the lesion.

17. The method of claims 1 and 9, wherein the active derivative is a recombinant form of the full length placental alkaline phosphatase.

18. The method of claim 17, wherein the active derivative has catalytic alkaline phosphatase activity and contains 80 to 100% of the sequence of the full length placental alkaline phosphatase.

19. The method of claim 2, wherein placental alkaline phosphatase or an active derivative thereof dispersed in a suitable carrier is topically administered onto a wound to reduce inflammation and associated pain caused by bacterial infection along with orally administered antibiotics as prescribed.

20. The method of claim 2, wherein placental alkaline phosphatase or an active derivative thereof dispersed in a suitable carrier is topically administered onto a wound not responding to antibiotics to reduce inflammation and associated pain caused by drug resistant bacterial infection.
